(19) United States
(12) Patent Application Publication
(10) Pub. No.: US 2006/0052327 A1
(43) Pub. Date: Mar. 9, 2006

(54) Cell specific gene silencing using cell-specific promoters in vitro and in vivo

(76) Inventors: Lin Liu, Stillwater, OK (US); Deming Gou, Stillwater, OK (US); Telugu Narasaraju, Stillwater, OK (US); Narendranath Reddy Chintagari, Stillwater, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 11/221,096

(22) Filed: Sep. 7, 2005

Related U.S. Application Data
(60) Provisional application No. 60/607,671, filed on Sep. 7, 2004.

(51) Int. Cl. A61K 48/00

(52) U.S. Cl. 514/44

(57) ABSTRACT

Cell-specific methods for silencing genes using double-strand inhibitory RNA (iRNA) are provided, as are constructs for carrying out the methods.

### REFERENCE TO RELATED APPLICATIONS

This application claims benefit of U.S. provisional patent application U.S. 60/607,671, filed Sep. 7, 2004, the complete contents of which are hereby incorporated by reference.

### STATEMENT REGARDING FEDERALLY SPONSORED RESEARCH

The Government of the United States of America may have certain rights in this invention pursuant to Grant No. 5 R01 HL52146-09 and Grant No. 1 R01 HL071628-01A1 awarded by the National Institute of Health.

### FIELD OF THE INVENTION

The invention relates to the use of double-strand inhibitory RNA (iRNA) to silence genes. In particular, the invention provides methods to use iRNA to silence genes in a cell specific manner.

### BACKGROUND OF INVENTION

RNA interference (RNAi) is a post-transcriptional process triggered by the introduction of double-stranded RNA, which leads to gene silencing in a sequence-specific manner (1). Specific gene silencing may be achieved in a variety of cell systems using chemically synthesized or in vitro transcribed small interfering RNA (siRNA) (2) as well as PCR or DNA vector-based short hairpin RNA (shRNA) (3-6). A few promoters have been reported to drive shRNA expression in cells, including RNA polymerase III-based promoters, U6 and H1, and RNA polymerase II promoter, CMV. However, the use of these promoters to drive shRNA expression in animals would silence a gene in all types of cells and thus produce undesirable effects in non-target cells. The lack of cell specificity limits the use of this technique in vivo. Cell-specific targeting of siRNA is an important issue to consider in RNAi therapeutics (7). If one wants to silence a gene in a particular type of cell without affecting its expression in other types of surrounding cells, a cell-specific promoter has to be used. So far, there are no successful reports in the use of a cell-specific promoter to drive shRNA expression in cells. Most recently, the use of tissue-specific recombination to produce tissue-specific knock-down has been reported (8).

The prior art has thus-far failed to provide methods that employ gene specific silencing that is cell-specific to treat conditions associated with the expression of a specific gene.

### SUMMARY OF INVENTION

The present invention provides methods that utilize the technique of RNA interference to bring about specific gene silencing in order to treat conditions associated with the expression of specific genes. However, in contrast to the prior art, the present methods do so in a cell-specific manner, i.e. the silencing of the targeted gene of interest occurs only (or at least primarily) in a single, targeted cell type. Thus, an undesirable feature of the prior art (silencing a targeted gene in all types of cells, which may produce undesirable effects in non-target cells) is eliminated. This is accomplished by creating constructs in which transcription of the silencing (interfering) RNA is driven by a promoter that is active only in the type of cell that is targeted (a cell specific promoter). Introduction of such a construct into a targeted cell will result in transcription of the RNA because the promoter will be activated inside the targeted cell. However, the same construct, when introduced into a different type of cell, will not be active, and the RNA will not be transcribed. Thus, the silencing RNA is produced only in cells in which the promoter that drives its transcription is active.

In particular, a strategy for specifically silencing genes in alveolar epithelial type II cells of mammalian lungs is described herein. Such a strategy allows the silencing of one or more genes in this type of cell in order to treat disease conditions that results from or are exacerbated by expression of the targeted genes. In addition, the ability to do so allows direct elucidation of the function of a targeted gene in targeted cells, thereby facilitating the development of more refined disease models. As is described in detail in the Examples section, advenoviral vectors containing various shRNA under the control of SP-C promoter were constructed. Using these vectors, specific silencing of target genes in type II cells, but not in other lung cells, was demonstrated both in vitro and in vivo. These results provide proof of principal of the inventive concept, and it could be expected that one of ordinary skill in the art could use the invention to target almost any particular cell or tissue type in vivo or in vitro, and to silence a gene in only that cell type using RNA silencing.

The invention provides a method for the treatment of conditions caused or exacerbated by expression of a specific gene by decreasing translation of mRNA encoded by the specific gene in a specific type of cell. The method includes the step of providing to the specific type of cell a vector comprising i) expressible DNA encoding RNA capable of forming a dsRNA structure, in which a nucleotide sequence of a portion of the dsRNA structure is identical to a nucleotide sequence of a portion of the mRNA; and ii) a promoter sequence operationally linked to the expressible DNA. The promoter sequence is active only in the specific type of cell (i.e. targeted operation is achieved by having the dsRNA formed in specific types of cells. In one embodiment, the specific type of cell is a lung cell, and in particular may be an alveolar Type II cell. However, cancer cells or other cells of interest can be subjected to targeted gene silencing according to this invention. In one embodiment, the promoter is SP-C. In one embodiment, the dsRNA structure is shRNA. In one embodiment, the vector is an adenoviral vector.

The invention further provides a vector which comprises: i) expressible DNA encoding RNA capable of forming a dsRNA structure, in which a nucleotide sequence of a portion of the dsRNA structure is identical to a nucleotide sequence of a portion of an mRNA of interest; and ii) a promoter sequence operationally linked to the expressible DNA, wherein the promoter sequence is active only in a specific type of cell. In one embodiment, the specific type of cell is a lung cell, and in particular may be an alveolar Type II cell. In one embodiment, the promoter is SP-C. In one embodiment, the dsRNA structure is shRNA. In one embodiment, the vector is an adenoviral vector.

The invention further provides a method of reducing expression of a specific gene by decreasing translation of mRNA encoded by the specific gene in lung cells or other cells in vivo in a patient in need thereof. The method comprises the step of administering to the lung cells a vector comprising i) expressible DNA encoding RNA capable of forming a dsRNA structure, in which a nucleotide sequence of a portion of the dsRNA structure is identical to a nucleotide sequence of a portion of the mRNA; and ii) a promoter sequence operationally linked to the expressible DNA, wherein the promoter sequence is active only in the lung cells. In one embodiment, the lung cell is an alveolar Type II cell. In one embodiment, the promoter is SP-C. In one embodiment, the dsRNA structure is shRNA. In one embodiment, the vector is an adenoviral vector.

### BRIEF DESCRIPTION OF THE DRAWINGS

FIG. 1. Schematic representation of the nucleic acid constructs of the invention.

FIG. 2. SP-C-driven shRNA silences exogenous EGFP expression in type II cells, but not in L2 cells. (A): A schematic illustration of the human SP-C promoter-driven shRNA adenoviral vector (Ad/SP-C-shRNA). The shRNA contains a sense strand (S), a 9-nt loop (L), and an anti-sense strand (AS), followed by TT and a 66 bp of minimal poly A (mA). (B) and (C): Effect of siRNA expressed from SP-C promoter constructs on EGFP expression on primary culture of alveolar type II cells (B) and L2 cells (C). The cells were infected with Ad/CMV-EGFP (a & d), Ad/CMV-EGFP and the siRNA control, Ad/SP-C-shCon (b & e), and Ad/CMV-EGFP and Ad/SP-C-shEGFP (c & f). After 5 days, the cells were examined with a fluorescence microscope (a-c), and the nuclei stained with DAPI to visualize the cells in the fields (d-f). Scale bar: 20 μm. (D) and (E): mRNA (D) and protein (E) levels of EGFP in type II and L2 cells as determined by RT-PCR and Western blotting. β-actin was used as a loading control. Upper panels show a representative gel or blot. Lane 1: Ad/CMV-EGFP; Lane 2: Ad/CMV-EGFP and Ad/SP-C-shCon; Lane 3: Ad/CMV-EGFP and Ad/SP-C-shEGFP. Lower panels: mRNA and protein levels were quantitated after being normalized with β-actin signals. The results are expressed as a percentage of control (Ad/CMV-EGFP). Data shown are means±S.E. (n=3). The asterisk denotes a P of <0.01 v.s. Ad/CMV-EGFP group.

FIG. 3. Analysis of shRNAs transcripts driven by SP-C promoter in type II cells. Type II cells cultured on air-liquid model were infected with Ad/SP-C-shCon (Lane 1) or Ad/SP-C-shEGFP (Lane 2) adenovirus for 5 days. After isolation of total RNA with TRI reagents, 20 μg of RNA were analyzed by Northern blot on a 15% polyacrylamide-urea gel. The blot was hybridized with ³²P-labeled sense shEGFP oligonucleotides (top panel). After being stripped, the same blot was re-probed by ³²P-labeled sense control (shCon) oligonucleotides (middle panel). RNA size markers are indicated on the left side of the gel. The hairpin RNAs were labeled on the right side. RNA quality were shown by the ethidum bromide staining of the 28S and 18S RNA (bottom panel).

FIG. 4. Specific silencing of EGFP and lamin A/C by SP-C-driven shRNA in a lung cell mixture. (A) EGFP: The mixed lung cells were infected with Ad/CMV-EGFP plus Ad/SP-C-shCon or Ad/SP-C-shEGFP adenoviruses for 5 days. a & e: EGFP fluorescence; b & f: immunostaining with anti-SP-C antibodies to identify type II cells. (B) lamin A/C: The mixed lung cells were infected with Ad/SP-C-shCon or Ad/SP-C-shLamin adenovirus for 5 days. The cells were double-labeled with anti-lamin A/C (a & e) to monitor the lamin A/C expression and anti-SP-C (b & f) antibodies to identify type II cells. Arrows: type II cells; stars: non-type II cells. Scale bar: 20 μm. (C) Quantitation of EGFP (top) or Lamin A/C (bottom) silencing in Ad/SP-C-shEGFP- or Ad/SP-C-shLamin-treated type II cells: Cell counting was performed based on the number of type II cells (red) with or without EGPF or lamin A/C expression (green). 80-100 type II cells were counted for each slide. The results were expressed as a percentage of control (Ad/CMV-EGFP/SP-C-shCon or Ad/SP-C-shCon). Data are means±S.E. (n=3). The asterisk denotes a P of <0.01 v.s. control group.

FIG. 5. Adenovirus-mediated annexin A2 gene silencing by SP-C-driven shRNA in in vitro lung organ culture. (A) Purified adenoviruses expressing annexin A2 shRNA or the control shRNA under the control of SP-C promoter (Ad/SP-C-shAII or Ad/SP-C-shCon) were used to infect in vitro lung organ culture. After 5 days, paraffin-embedded sections from cultured lung organs were double-immunostained with anti-annexin A2 (a & e) and anti-SP-C (b & f) antibodies. The merged images and bright fields are shown in c & g and d & h, respectively. The upper right corners show the enlarged images. Scale bar: 20 μm. (B) Quantitation of annexin A2 silencing in type II cells: Cell counting was performed based on the number of type II cells (red) with or without EGPF or lamin A/C expression (green). 80-100 type II cells were counted for each slide. The results were expressed as a percentage of control (Ad/SP-C-shCon). Data shown are means±S.E. (n=3).

FIG. 6. Adenovirus-mediated annexin A2 gene silencing by SP-C-driven shRNA in rat lungs. Purified adenoviruses (5×10¹¹ particles) expressing annexin A2 shRNA or the control shRNA under the control of SP-C promoter (Ad/SP-C-shAII or Ad/SP-C-shCon) were directly delivered into rat lungs. After 5 days, paraffin-embedded sections from infused lung were double-immunostained with anti-annexin A2 (a & e) and anti-SP-C (b & f) antibodies. The merged images and bright fields are shown in c & g and d & h, respectively. The upper right corners show the enlarged images. Scale bar: 20 μm.

### DETAILED DESCRIPTION

The present invention provides methods of gene silencing by RNA interference that are cell-specific. That is, the invention can eliminate or reduce the expression of a specific gene in a specific type of cell or tissue or interest, in vitro and in vivo. The method involves creating constructs that encode the interfering (silencing) RNA in which a promoter that is active in only one type of cell mediates transcription of the RNA. Thus, when the construct is administered to an individual, even though the construct may enter many different types of cells in the individual, the RNA will be produced only in the one type of cell in which the promoter is active.

The methods of the present invention involve the silencing of a specific gene (a “gene of interest” or “targeted gene” or “selected gene”). By “silencing” a gene, we mean that expression of the gene product is reduced or eliminated, in comparison to a corresponding control gene that is not being silenced. Those of skill in the art are familiar with the concept of comparing results obtained with control vs. experimental results. Without being bound by theory, it is believed that RNAi is characterized by specific mRNA degradation after the introduction of homologous double stranded RNA (dsRNA) into cells. The dsRNA is recognized and processed into small interfering RNAs (siRNAs) of 19-25 nucleotides in length by an endonuclease enzyme dimer termed Dicer (RNase III family). These siRNAs, in turn, target homologous RNA for degradation by recruiting the protein complex, RNA-induced silencing complex (RISC). The complex recognizes and cleaves the corresponding mRNA (Dykxhoom D M, Novina C D and Sharp P A, Nature Review, 4: 457-467, 2003; Mittal V, Nature Reviews, 5: 355-365, 2004).

In general, “reduced or eliminated” refers to a reduction or elimination of detectable amounts of the gene product by an amount in the range of at least about 10% to about 100%, or preferably of at least about 25% to 100%, or more preferably about 50% to about 100%, and most preferably from about 75% to about 100%. If desired, a reduction or elimination may be determined by any of several methods that are well known to those of skill in the art, and may vary from case to case, depending on the gene that is being silenced. For example, such a reduction or elimination of the expression of the gene may be determined by quantification of the gene product (e.g. by determining the quantity of a protein, polypeptide or peptide that is made) or quantification of an activity of the gene product (e.g. an activity such as enzymatic activity, signaling or transport activity, activity as a structural component of the cell, activity to change cell behaviors, activity to kill bacteria or viruses, activity to induce gene expression, etc.), or by observation and quantification of a phenotypic characteristic of the targeted cell in comparison to a control cell (e.g. lack of ability to proliferate, differentiate, or undergo apoptosis, etc). Any suitable means to determine whether or not a targeted gene has been silenced may be used. Further, the result of silencing of the gene in a cell may be highly variable, e.g. the cell may die, or become quiescent; the metabolism of the cell may be altered; the cell may lose the ability to metastasize; etc. The specific effect of silencing the gene is not a key feature of the invention, so long as the effect results in a desired outcome (e.g. ameliorating an undesired condition, or bringing about a desired condition, in the cell).

The constructs utilized in the practice of the invention include at least one cell-specific promoter that is operationally linked to nucleotides (usually DNA) encoding an RNA molecule. By “operationally linked” we mean that, in the vector, the promoter is associated with the nucleotides encoding the RNA in a manner that allows the promoter to drive transcription (i.e. expression) of the RNA from the nucleotides. Transcription of RNA from, e.g. a DNA template is well-understood. Those of skill in the art will recognize that many such cell-specific promoters are known, and additional cell-specific promoters are continually being discovered. All such cell-specific promoters are encompassed by the present invention.

The promoters that are employed in the invention are cell-specific. Those of skill in the art will recognize that some tissues are made up of a single type of cell, or some types of cells are expressed only in a particular tissue, and thus, the promoter may be referred to as a “tissue-specific” promoter. In addition, some promoters may be specific for more than one, but not all, cells. These promoters may also be used in the practice of the invention, so long as it is desired to silence a gene in all cells in which the promoter is active. Examples of cell (or tissue)-specific promoters and the cells for which they are specific include but are not limited to
SP-C and SP-B promoter: lung epithelial type II cells
Aquaporin 5 promoter; lung epithelial type I cells
CCSP promoter; lung Clara cells
Cytokeratin 18 (K18) promoter; lung epithelial cells
Vascular endothelial growth factor receptor type-1 (flt-1) promoter: cells
FOXJ1 promoter; lung airway surface epitheilium.
Tie2 promoter, lung endothelial cells
Pre-proendothelin-1 (PPE-1) promoter, endothelial cells
Albumin promoter, liver
MCK promoter, muscle
Myelin basic protein promoter, oligodendrocytes glial cells
Glial fibrillary acidic protein promoter, glial cells
NSE promoter, neurons
KDR, E-selectin, and Endoglin promoters, tumour endothelium
Telomerase reverse transcriptase promoter; cancer cells.
Carcinoembryonic antigen (CEA) promoter; lung, breast, colon cancers
Alpha-ftoprotein (AFP) promoter; hepatocellular carcinoma (HCC)
ErbB2 promoter, breast cancer
Tyrosinase gene promoter, melanoma
Prostate-specific antigen (PSA) promoter, prostate-specific
Muc-1 promoter, breast cancer
Osteocalcin promoter, osteosarcoma
Secretory leukoprotease inhibitor, ovarian, cervical carcinoma
HRE promoter, solid tumours

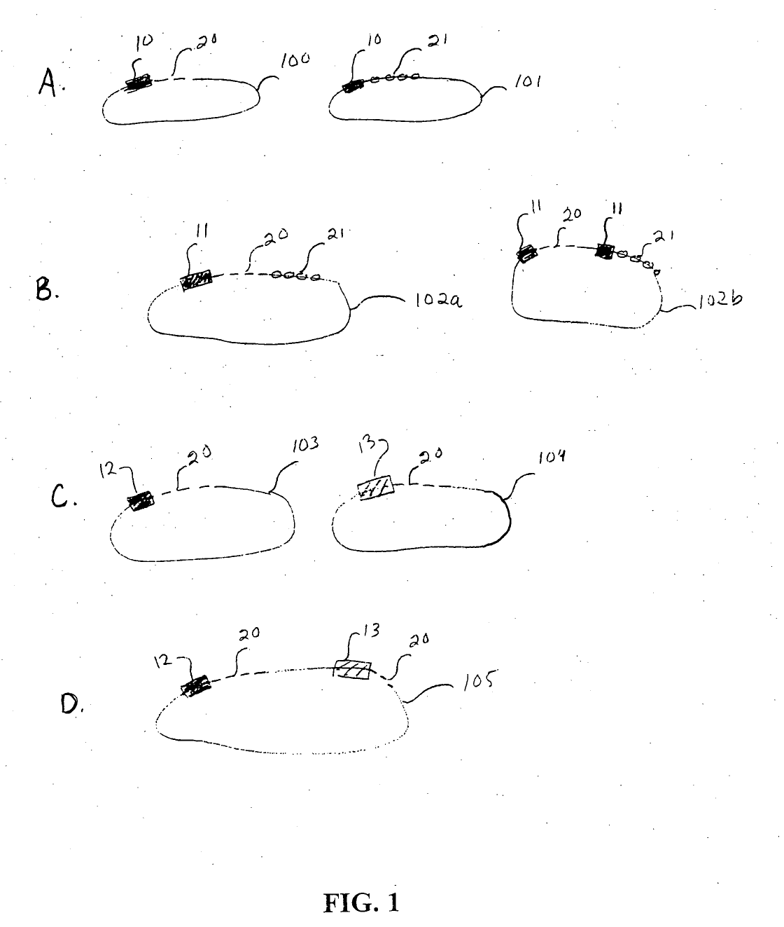

The RNA molecule that is encoded by the construct of the present invention ultimately forms a double-strand RNA molecule within the cell in which it is transcribed. In general, one strand of the double-strand RNA structure will be in the range of from about 10 to about 30 ribonucleotides in length, and preferably from about 19 to about 25 ribonucleotides in length. Those of skill in the art will recognize that several viable strategies exist for forming such double-strand RNA. For example, a single RNA molecule that includes two regions that are homologus to each other and that will thus hybridize may be utilized. In this case, a hairpin loop will be formed. Alternatively, two separate RNA segments that are homologus to each other and that will thus hybridize may be formed. Other alternatives include microRNA-based hairpin RNA, etc. In one embodiment of the invention, only one gene is silenced in a particular, targeted cell type. However, this need not be the case. For example, provision of multiple constructs with the same cell-specific promoter but which encode different silencing RNAs may be used within the practice of the invention. This is illustrated in FIG. 1A, which shows constructs 100 and 101, both of which contain promoter 10. However, the nucleic acid that is expressibly linked to promoter 10 differs between the two, construct 100 containing nucleic acid sequence 20, and construct 101 containing nucleic acid 21. This may be advantageous in the case where a condition is known to result from the expression of two (or more (e.g. up to four or more) genes in a specific type of cell. Further, it should be possible to express more than one silencing RNA in a single construct, driven by a single cell-specific promoter, or by more than one promoter arranged in tandem (e.g. two or more promoters). Thus, the invention contemplates using a single construct for silencing more than one gene. This embodiment is illustrated in FIG. 1B, where construct 102a contains promoter 11, which drives expression of both nucleic acid sequence 20 and nucleic acid sequence 21. Alternatively, as shown by construct 102b, the single construct may contain multiple copies of a single promoter 11 driving expression of two (or more) different sequences, 20 and 21. In addition, the invention also contemplates targeting more than one cell type at a time by administering together multiple constructs that differ in targeting characteristics, i.e. constructs that differ in that they contain different cell-specific promoters. This embodiment is illustrated in FIG. 1C, where constructs 103 and 104, both of which contain nucleic acid 20, but which contain different promoters 12 and 13, each of which is specific for a particular cell or tissue type. In this manner, the same gene could be targeted in different cell types. Alternatively, a single construct may contain more than one (e.g. up to four or more) cell-specific promoters operationally linked to a silencing RNA. This embodiment is illustrated in FIG. 1D, where construct 105 contains two separate promoters 12 and 13, each of which is specific for a different cell/tissue type, and each of which drive transcription of nucleic acid sequence 20. In this case, the RNA (or RNAs) encoded by the construct will be expressed in each type of cell for which a cell-specific promoter has been included in the construct. In any case, the silencing RNAs encoded by the construct will still not be expressed in every cell that takes up the construct, but only in cells in which the cell-specific promoter is active.

In one embodiment of the invention, the promoter that is used is a constitutive promoter. However, in another embodiment, the promoter that is utilized is an inducible promoter. In this case, the formation of the silencing dsRNA in a targeted cell is not only cell specific, but expression of the RNA is activated or induced by a signal from the environment. Those of skill in the art will recognize that many suitable inducible promoters exist that could be used in the practice of the invention, examples of which include but are not limited to: (1) tetracycline-inducible system: The shRNA expression is under the control of the modified U6, H1, or 7SK promoter, in which the tetracycline operator (TetO) sequence is added. The tetracycline repressor (tTR) or tTR-KRAB expression is under the control of cell-specific promoter, such as SP-C promoter. In the absence of an inducer, the tTR or t-TR-KRAB binds to TetO and inhibits the expression of shRNA. The addition the inducer, doxycycline (DOX) removes the tTR or tTR-KRAB from the TetO and thus induces the transcription of shRNA in a cell-dependent manner since tTR or tTR-KRAB is only expressed in a specific cell type. (2) IPTG-inducible system. This is similar to (1) above except that TetO and tTR are replaced with lac operator and lac repressor, respectively. The inducer in this case is isopropyl-thio-beta-D-galactopyranoside (IPTG). (3) CER inducible system: a neomycin cassette (neo) is inserted into the U6 or H1 promoter that drives shRNA expression. The insertion disrupts the promoter activity and thus no transcription of shRNA occurs. However, the cell-specific expression of Cre recombinase under the control of a cell-specific promoter restores the promoter activity and thus the expression of shRNA in a specific cell type. The inducer in this case is tamoxifen. (4) Ecdysone-inducible system. The inducible ecdysone-responsive element/Hsmin (ERE/Hsmin) is added to U6 promoter that controls the expression of shRNA. The expression of two proteins, VgEcR and RXR are driven by cell-specific promoters. In the presence of the inducer, MurA, VgEcR and RXR form a dimer and bind to ERS/Hsmin to initiate the transcription of shRNA in a specific cell type. It will be understood that a construct can have more than one constitutive promoter, as well as combinations of constitutive and inducible promoters.

The methods of the invention involve creating constructs (e.g. vectors) that contain at least one cell-specific promoter that is operationally connected to DNA that encodes RNA for silencing a specific gene. In addition, the constructs are suitable for administration to individuals that are to be treated by the methods. In a preferred embodiment of the present invention, the construct is an adenoviral vector for delivery as disclosed herein. However, those of skill in the art will recognize that many other systems for delivering a nucleic acid to cells already exist or are currently under development, and would be suitable for use in the practice of the present invention. For example, other vectors (both viral and non-viral) may be utilized (e.g. plasmids, viral particles, baculovirus, phage, phagemids, cosmids, phosmids, bacterial artificial chromosomes, viral DNA, P1-based artificial chromosomes, yeast plasmids, and yeast artificial chromosomes, and the like. Some forms of viral vectors may be especially useful (e.g. viral vectors such as retrovirus, lentivirus, adenovirus or adenovirus-associated vectors). Alternatively, the construct may be delivered via liposomes or liposome-type delivery systems, or via attenuated bacterial delivery systems, by binding (either covalently or non-covalently) to another molecule which enhances delivery, by direct injection of the construct, or by catheterization, and the like. Further, other procedures which enhance the delivery of nucleic acids into cells may be utilized in conjunction with the practice of the present invention, e.g. various means of altering cell membrane permeability (e.g. ultrasound, exposure to chemicals or membrane permeability altering substances, and the like). Any appropriate means of delivery of the construct may be utilized in the practice of the present invention.

The present invention also provides a therapeutic composition comprising an effective dose of construct as described herein. The construct may conveniently be provided in the form of formulations suitable for administration to mammals. In addition, a suitable administration format may be determined by a medical practitioner for each patient individually. Suitable pharmaceutically acceptable carriers (e.g. aqueous, oil-based, etc.) and their formulation are described in standard formulations treatises, e.g., Remington's Pharmaceuticals Sciences by E. W. Martin. See also Wang, Y. J. and Hanson, M. A. “Parental Formulations of Proteins and Peptides: Stability and Stabilizers”, Journals of Parental Sciences and Technology, Technical Report No. 10, Supp. 42:2S (1988). Constructs of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions may also be prepared to enhance shelf life and stability. The therapeutically useful compositions for use in the practice of the invention are prepared by mixing the ingredients following generally accepted procedures. For example, the selected components may be mixed to produce a concentrated mixture which may then be adjusted to the final concentration and viscosity by the addition of water and/or a buffer to control pH or an additional solute to control tonicity.

In a preferred form for use by a physician, the compositions will be provided in dosage form containing an amount of a construct that will be effective in one or multiple doses to induce RNA silencing. As will be recognized by those in the field, an effective amount of therapeutic agent will vary with many factors including the age and weight of the patient, the patient's physical condition, the type of condition being treated, and other factors.

The effective dose of the constructs of this invention will typically be in the range of about 10⁷ to about 10¹² pfu (plaque forming units).

The delivery of the constructs may be in general local or systemic, and may be accomplished by a variety of methods, including but not limited to injection, positive pressure, continuous flow infusion, oral or intravenous administration, inhalation, and the like. Any suitable delivery means may be utilized in the practice of the present invention. Further, the constructs may be delivered in conjunction with other therapies.

The methods of the invention can be used to treat conditions that are caused at least in part by the expression of a particular gene. In general, conditions that are treated by the methods of the invention are those in which the phenotypic expression of the targeted gene would generally be considered unfavorable or untoward for the individual in whom the gene is expressed. For example, the expression of the gene may lead to or contribute to the development of symptoms of a disease, or may predispose an individual in whom the gene is expressed to the development of such symptoms. Such conditions include but are not limited to:
Cancer, e.g. lung cancer, leukemia and lymphoma, pancreatic cancer, colon cancer, prostate cancer, glioblastoma, ovarian cancer, breast cancer, head and neck cancer, liver cancer, skin cancer, uterine cancer;
for which potential target genes (i.e. genes in the cell or tissue type that will be silenced) include: BCR/ABL fusion protein, K-RAS, H-RAS, bcl-2, Bax, FGF-4, Skp-2, CEACAM6, MMP-9, Rho, spingosine-1 phosphate-R, EGF receptor, EphA2, focal adhesion kinase, surviving, colony-stimulating factor, Wnt, PI3 kinase, Cox-2, H-Ras, CXCR4, BRAF, Brk, PKC-alpha, telomerase, myc, ErbB-2, cyclin D1, TGF-alpha, Akt2,3, α6β4 integrin, EPCAM receptor, androgen receptor, and MDR.
Infectious diseases, e.g. HIV, Hepatitis B and C, Respiratory syncytial virus, inflenza, West Nile virus, Coxsakievirus, severe acute respiratory syndrome (SARS), cytomeglovirus, Paillomomavirus, poliovirus, Rous sarcoma virus, Rotavirus, Adenovirus, Rhinovirus, Poliovirus, Malaria (parasites);
for which potential target genes include: viral genes or host receptors (CCR5, CD4, HB surface antigen, viral genes, CD46, PP1).
Ocular diseases, e.g. age-related macular degeneration, herpetic stromal keratitis, diabetic retinopathy;
for which potential target genes include: VEGF, VEGF receptor, and TGF-beta receptor.
Neurological diseases e.g. amyotrophic lateral sclerosis, Alzheimer's disease, myastenic disorders, Huntingon's disease, Spinocerebellar ataxia;
for which potential target genes include: SOD1, Beta-secretase (BACE1), SCCMS, Huntingin, Ataxin 1.
Respiratory diseases, e.g. asthma, Chronic obstructive pulmonary diseases (COPD), cystic fibrosis, acute lung injury;
for which potential target genes include: TGF-alpha, TGF-beta, Smad, CFTR, MIP-2, keratinocyte-derived chemokine (KC).
Other conditions or disorders, e.g. Metabolism diseases (obesity, cholesterol), inflammation (Rheumatoid arthritis), Hearing (autosomal dominant) etc.;
for which potential target genes include: AGRP, Apo B, TNF-alpha, Gap junction beta2.

Those of skill in the art will recognize that the RNAi technology of the present invention can be used to treat any condition for which it is desired to reduce or eliminate the expression of a particular gene or genes in a particular type of cell or cells. (See, for example, Uprichard S L, FEBS Letters, in press (2005), available on-line, Leung R K M, Whittaker, P A, Pharmacology & Therapeutics, 107: 222-239 (2005); Shankar P, Manjunath N, and Lieberman, J. JAMA, 293: 1367-1373, 2005)

However, the genes that are silenced (i.e. those whose expression is eliminated or reduced) need not be directly related to recognized “diseases” as such. For example, in some cases it may be desirable to silence genes related metabolism (e.g. to bring about weight loss, to lower cholesterol, etc.).

In one embodiment of the invention, the genes that are targeted for silencing are located in lung cells, i.e. the targeted or selected cells are lung cells. The lung is one of the major targets for gene therapy. Alveolar epithelium is composed of morphologically and functionally distinct type I and type II cells. A number of genes that are expressed in a highly cell-selective manner in the respiratory epithelium have been isolated and characterized. These include the genes encoding surfactant protein (SP)-A, B, and C, and the Clara cell secretory protein. Among them, SP-C is exclusively expressed in alveolar type II cells of the distal airways. The human SP-C promoter has been successfully used to express a transgene in a cell-specific manner (9). This, in a preferred embodiment of the invention, the cells that are targeted are alveolar type II lung cells, the gene that is targeted for silencing is a gene that is expressed in an alveolar type II cells lung cells, and the promoter that drives expression of the silencing RNA is one that is active only in alveolar type II lung cells, such as the SP-C promoter.

The present invention also has useful applications as a laboratory tool. The ability to selectively silence a single gene, or specific combinations of genes, within a particular cell type allows the elucidation of the function of a specific gene (or specific combination of genes) in the cell type. The ability to do so provides a useful tool for understanding the role of specific genes in cellular metabolism, in susceptibility to disease, disease progression, or other possible functions of the gene.

### EXAMPLES

Example 1. RNA interference (RNAi) is sequence-specific post-transcriptional gene silencing. Although it is widely used in the loss-of-function studies, none of current RNAi technologies can achieve cell-specific gene silencing. Here, we report a cell-specific RNAi system using an alveolar epithelial type II cell—specific promoter: surfactant protein C (SP-C) promoter. We show that the SP-C-driven small hairpin RNAs specifically depress the expression of exogenous reporter (enhanced green fluorescent protein) and endogenous genes (lamin A/C and annexin A2) in alveolar type II cells, but not other lung cells using in vitro cell and organ culture as well as in vivo. The present study provides an efficient strategy in silencing a gene in one type of cell without interfering with other cell systems and may have a significant impact on RNAi therapy.

Materials and Methods

Design and construction of shRNA and viral vectors: A DNA fragment containing 3.7-kb of human SP-C promoter, rtTA coding sequence and 0.45-kb SV40 poly A was PCR amplified with Pfu DNA polymerase (Stratagene, La Jolla, Calif.) and the primer pair, 5′-CACCTCGAGATCGATGAAGACTGCTGCTCTCTACCACGTT-3′ (SEQ ID NO: 1) and 5′-TTCGAACGCGTAATTCGAGCTCGGTACCCGGGGATCAGACATGA-3′ (SEQ ID NO: 2) from pSP-C-rtTA vector (9). pENTR/SP-C-rtTA vector was obtained by directionally cloning the purified PCR product into pENTR/D-Topo vector (Invitrogen, Carlsbad, Calif.). Later, pENTR/SP-C-rtTA vector was digested by Sal I and EcoR I restriction enzymes to remove the rtTA fragment between SP-C promoter and poly A sequences. The annealed shRNAs with Sal I-EcoR I overhangs were then cloned into SP-C vector through Sal I-EcoR I sites to obtain a new vector, pENTR/SP-C-shRNA-pA with a poly A terminal sequence. We replaced the poly A sequence with a minimal poly A (mA) at the EcoR I and BamH I restriction sites using two annealed oligonucleotides:

(SEQ ID NO: 3)

5′AATTCGAATTCAATAAAGGATCCTTTATTTTCATTGGATCCGTGTGTT
GGTTTTTTGTGTCTCGAG-3′
and

(SEQ ID NO: 4)

5′GATCCTCGAGACACAAAAAACCAACACACGGATCCAATGAAAATAAAG
GATCCTTTATTGAATTCG-3′

This minimal poly A sequence has been successfully used for CMV-driven shRNA (3). The shRNAs targeted to enhanced green fluorescent protein (EGFP), lamin A/C (Lamin), annexin A2 (AII), and an unrelated siRNA negative control (Con) contain a sense strand siRNA 19- or 21-nucleotide sequence, followed by a short spacer (5′-TTCAAGAGA-3′), an antisense strand, and two thymidines. Four sets of oligonucleotides with Sal I and EcoR I overhangs were synthesized:

1, shEGFP:

| | | |
|---|---|---|
| Top: | 5′TCGACCACAAGCTGGAGTACAACT | (SEQ ID NO: 5) |
| | ACTTCAAGAGAGTAGTTGTACTCCAG | |
| | CTGTGTTG-3′, | |
| Bottom: | 5′AATTCAACACAAGCTGGAGTACAA | (SEQ ID NO: 6) |
| | CTACTCTCTTGAAGTAGTTGTACTCC | |
| | AGCTTGTGG-3′, | |

2, shLamin:

| | | |
|---|---|---|
| Top | 5′TCGACCTGGATTTCCAGAAGAACA | (SEQ ID NO: 7) |
| | TTCAAGAGATGTTCTTCTGGAAATCC | |
| | AGTTG-3′, | |
| Bottom | 5′-AATTCAACTGGATTTCCAGAAGA | (SEQ ID NO: 8) |
| | ACATCTCTTGAATGTTCTTCTGGAAA | |
| | TCCAGG-3′, | |

3, shAII,

| | | |
|---|---|---|
| Top | 5′TCGACCGCATTGAAACAGCAATCA | (SEQ ID NO: 9) |
| | AGTTCAAGAGACTTGATTGCTGTTTC | |
| | AATGTTG-3′, | |
| Bottom | 5′-AATTCAACATTGAAACAGCAATC | (SEQ ID NO: 10) |
| | AAGTCTCTTGAACTTGATTGCTGTTT | |
| | CAATGCGG-3′, | |
| and | | |

4, shCon,

| | | |
|---|---|---|
| Top | 5′-TCGACTTCTCCGAACGTGTCACG | (SEQ ID NO: 11) |
| | TTTCAAGAGAACGTGACACGTTCGGA | |
| | GAATTG-3′, | |
| Bottom | 5′-AATTCTCCGAACGTGTCACGTTC | (SEQ ID NO: 12) |
| | TCTTGAAACGTGACACGTTCGGAGAA | |
| | G-3′. | |

All the shRNA sequences were subcloned into the pENTR vector with SP-C promoter and the minimal poly A sequences between the Sal I-EcoR I sites. The final clones were verified by DNA sequencing. The CMV-driven EGFP expression cassette was PCR-amplified using pEGFP-N1 (Clontech, Palo Alto, Calif.) as a template, and cloned into pENTR/D-Topo vector. All the inserts in pENTR vector were switched into the adenoviral vector, pAd/PL-DEST, through the Gateway technique (Invitrogen, Carlsbad, Calif.). The resulting adenoviral plasmids (Ad/SP-C-shEGFP, Ad/SPC-shLamin, Ad/SP-C-shAII, and Ad/CMV-EGFP) were linearized by Pac I and purified with GENECLEAN Turbo kits (Qbiogene, Carlsbad, Calif.). Using Lipofectamine 2000 (Invitrogen, Carlsbad, Calif.) as a transfection reagent, Pac I-linearized adenoviral plasmids were transfected into 293A cells for the generation of adenovirus. The adenoviruses were concentrated and purified by a cesium chloride density gradient ultracentrifugation (10). Infectious units and particle titers were determined by plaque assay and OD₂₆₀ (11).

Cell culture and adenoviral infection: Alveolar type II cells were isolated from rat lungs as previously described (12). A mixed lung cell preparation was obtained by using elastase digestion (4.5 units/ml) as previously described (13). The resulting cell mixture consisted of alveolar type I cells, type II cells, Clara cells, ciliated airway epithelial cells, fibroblasts, macrophages, and lymphocytes. To preserve type II cell phenotype, type II cells and mixed lung cells were cultured on an air-liquid culture system as previously described (14). The cells (3×10⁶) were plated on a 30 mm filter insert (Millipore, Bedford, Mass.) coated with rat-tail collagen and Matrigel (4:1, vol/vol, Collaborative Biomedical Products, Bedford, Mass.). One ml of DMEM containing 5% rat serum, 10 ng/ml keratinocyte growth factor, and 10 nM dexamethasone were added to each side of the insert. The plates were placed on a rocking rotator inside an incubator with 5% CO₂. After a 20-h culture, 0.3 ml of the same medium containing adenoviruses (400 mutiplicities of infection or MOI) was added to the apical surface, and 1.5 ml of the medium was placed on the outside of the insert. A 1:8 ratio was used when Ad/CMV-EGFP and Ad/SP-C-shRNA adenovirus were added simultaneously. The medium was changed on alternate days. On the 6^{th} day, the type II cells were directly examined for the EGFP fluorescence, and the mixed cells collected by disrupting the collagen gel with a pipette and filtering through 160-μm pore-size filter. Cells were cytospinned to coverslips for immunocytochemistry

In vitro lung organ culture: The lungs were excised from three-day-old rat pups and cut transversely into 5-mm slices using a sterile blade. The lung slices were cultured on a 30 mm filter insert. Serum free, hormone-free BGJb medium (0.3 ml) containing 0.2 mg/ml ascorbic acid, 0.5 units/ml penicillin, and 0.5 g/ml streptomycin was added on the inside of insert and 1.5 ml outside of insert (15). In this design, the lung slices were situated just above the median and remained suffused with medium by capillary action through the membrane. The adenovirus (6×10⁹ particles) was added on the inside of the insert at the time of plating. After culturing for 6 days, the organ culture was fixed with 4% (w/v) formaldehyde, embedded in paraffin, sectioned, and immunostained with anti-annexin II and anti-SP-C antibodies.

Adenoviral delivery into the rat lungs: Adult male Sprague-Dawley rats (200-250 g) were used for in vivo studies. Oklahoma State University Animal Use and Care Committee approved the animal procedures. Endotracheal intubation and administration of the virus was done as described earlier (16). In brief, the animals were anesthetized with an intraperitoneal injection of Ketamine and Xylazine. The epiglottis and trachea of the animal were visualized using a modified intubation wedge. The animals were then orally intubated using a sterile 18-guage intravenous catheter. Immediately prior to the administration of the adenovirus, the animals were forced to exhale by circular compression of the thoracic cavity and then 200-400 μl of the adenovirus (5×10¹¹ particles) in phosphate buffered saline containing 50% Survanta (Abbot laboratories, Columbus, Ohio), 1 mg/ml of protamine sulfate, and 250 μg/ml hydrocortisone (17;18) was administered. The virus was incubated for 10 min with protamine sulfate before being administrated into the animal. Animals were sacrificed on the 5^{th} day. The lungs were instilled with 4% formaldehyde (w/v) in phosphate buffered saline (pH 7.4), embedded in paraffin, and processed for immunostaining.

Northern Blotting: Total RNA was isolated with TRI reagents (Molecular Research Center, Inc., Cincinnati, Ohio) from the cultured type II cells. RNA (20 μg/lane) were electrophoretically separated on a 15% polyacrylamine-7 M urea gel and transferred by electroblotting onto to Hybond N+ membrane (Amersham Pharmacia Biotech). The sense oligonucleotides (100 pmoles) of shEGFP or shCon were end-labeled with polynucleotide kinase and [³²P]-ATP (150 μCi), purified through a G-25 MicroSpin Column (Amersham Pharmacia Biotech), heated for 5 min to 65° C., and then used for hybridization at 37° C. overnight. Membrane was washed for 2 times of 5 min interval at room temperature in 2×SSC plus 0.1% SDS, 3 times for 10 min in 0.1×SSC plus 0.1% SDS, and exposed on BioMax MS films (Kodak).

Reverse Transcription/Polymerase Chain Reaction: After being treated with DNase, 1 μg of total RNA was reverse-transcribed into cDNA using M-MLV reverse transcriptase (200 U) in presence of 100 ng 18-mer oligo(dT) and 5 ng EGFP reverse primer. One μl of cDNA were used to amplify the EGFP fragment with 1× PCR buffer, 1.5 mM MgCl₂, 200 μM of each dNTP, 200 μM of primers, and Taq DNA polymerase. For normalization of RNA loading, the housekeeping gene, β-actin, was also amplified from each sample. The primer sequences are as follows: EGFP, forward, 5′-TGCCACCTACGGCAAGCTGA-3′ (SEQ ID NO: 13) (111-130), reverse, 5′-TCGATGTTGTGGCGGATCTT-3′ (SEQ ID NO: 14) (499-518); β-actin, forward, 5′-GGCATTGTAACCAACTGGGACGATATG-3′ (SEQ ID NO: 15) (220-246), reverse, 5′-TTCATGGATGCCACAGGATTCC-3′ (SEQ ID NO: 16) (807-828). PCR amplification was performed using the following conditions: 1 cycle of 95° C. for 3 min, 25 cycles of 94° C. for 30 sec, 56° C. for 1 min, and 72° C. for 1 min, followed by a final elongation step of 72° C. for 7 min. After amplification, 10 μl aliquots of PCR products from each condition were separated on a 1.5% agarose gel. Signals were quantified by densitometric analysis using Bio-Rad Quantity One 4.0.3 software.

Western blotting: Cells were lyzed at 4° C. for 1 h in the lysis buffer (50 mM Tris-HCl, pH 7.4, 250 mM sucrose, 1% Triton X-100, 10 mM EGTA, 2 mM EDTA, 20 μg/ml leupeptin and 1 mM phenylmethylsulfonyl fluoride). Ten to twenty μg of the proteins were resolved on 12% sodium dodecyl sulfate (SDS)-polyacrylamide gel electrophoresis (PAGE) under reducing conditions and electrophorically transferred onto a nitrocellulose membrane. The membranes were blocked with Tris-buffered saline plus 0.1% Tween 20 (TTBS) containing 5% non-fat milk for 1 h, incubated with the appropriate primary antibodies (anti-β-actin, 1:4000 dilution; anti-GFP, 1:1000 dilution) in TTBS containing 1% BSA for 2 h, followed by incubation with secondary antibodies (horseradish peroxidase-conjugated IgG, 1:5,000 dilution) for 1 h. Finally, the proteins were visualized by enhanced chemiluminescence reagents. Signals were quantified by densitometric analysis using Bio-Rad Quantity One 4.0.3 software.

Immunostaining: Immunohistochemistry and immunocytochemistry were performed as described previously (19). The primary and secondary antibodies were: polyclonal goat anti-SP-C (1:50), monoclonal anti-lamin A/C (1:50), and polyclonal rabbit anti-annexin II (1:50) antibodies (all from Santa Cruz Biotechnology, Santa Cruz, Calif.) and Alexa 488 or Alexa 546 -conjugated anti-goat (1:200), Alexa-488-conjugated anti-mouse (1:200), Cy 3-conjugated anti-mouse (1:200), and Alexa 546-conjugated anti-rabbit (1:200) IgG (Molecular Probes, Eugene, Oreg.).

Results and Discussion

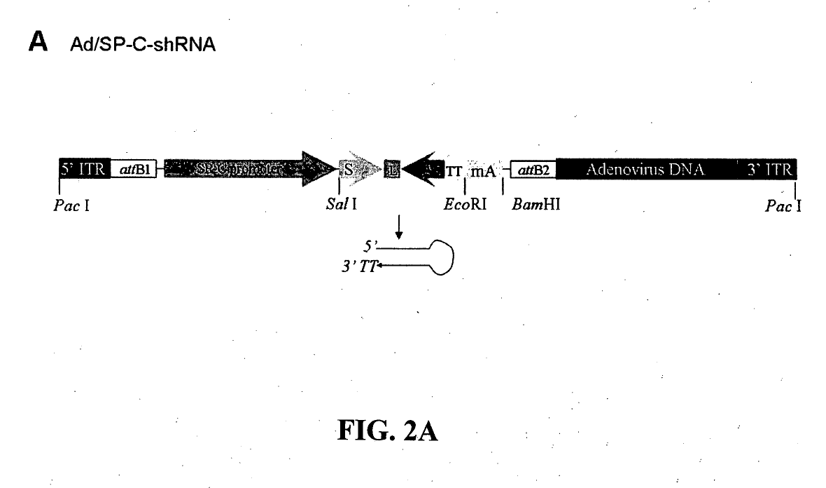

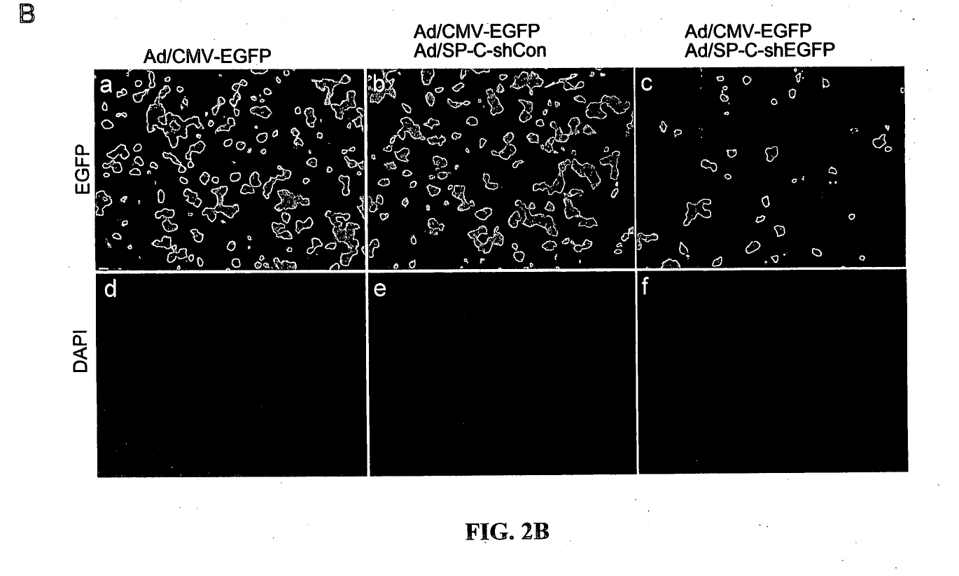

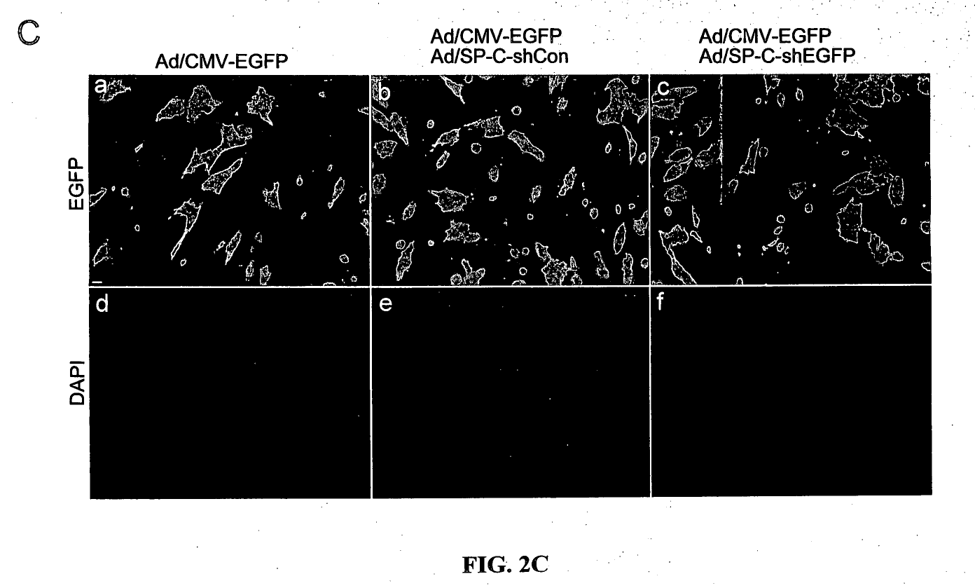

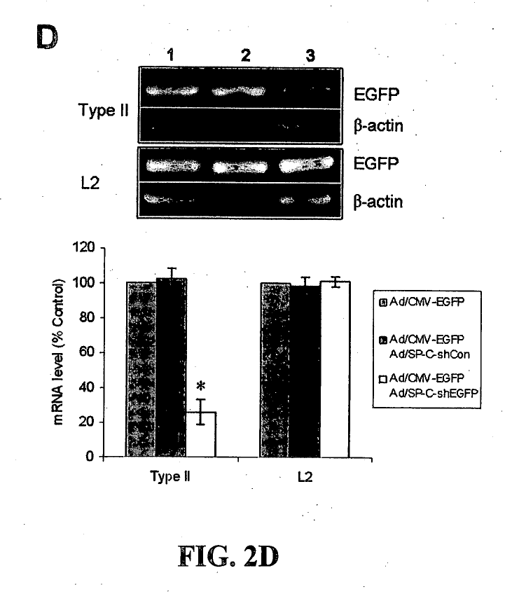

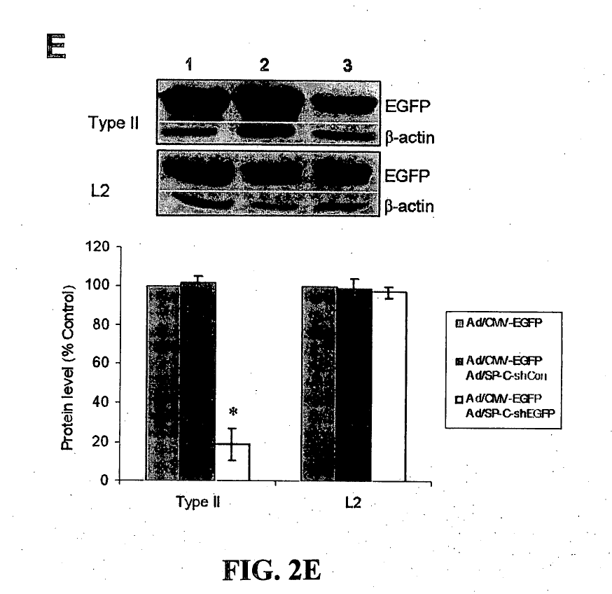

To determine whether the SP-C promoter can be used to produce siRNA and silence gene expression in a cell-specific fashion, we constructed an adenoviral vector, in which shRNA in the form of a small hairpin structure, was placed under the control of the 3.7-kb human SP-C promoter, and followed by a minimal poly A (FIG. 2A). The minimal poly A has successfully been used for CMV-driven shRNA (3). We chose the adenoviral vector because of its high transfection efficiency in primary culture cells and animals. We first tested whether SP-C-driven shRNA depressed the expression of the exogenous reporter gene, EGFP, in primary culture of alveolar type II cells. It is well known that type II cells lose their phenotype and SP-C expression, and trans-differentiate into type I—like cells when cultured on plastic dishes. We therefore used an air-liquid culture system that mimics the in vivo conditions expected in the lung. This system has been reported to maintain the type II cell phenotype including the expression of SP-C (14). The exogenous EGFP expression in type II cells was obtained with a CMV-EGFP adenoviral vector, containing EGFP under the control of the CMV promoter. The infection of Ad/CMV-EGFP adenovirus alone resulted in a high expression of EGFP in isolated type II cells by a direct visualization under a fluorescence microscope (FIG. 2B a). EGFP expression was markedly reduced when type II cells were co-infected with Ad/CMV-EGFP and SP-C-driven shRNA targeted to EGFP (Ad/SP-C-shEGFP) adenoviruses (FIG. 2B c). Inhibition was sequence-specific because the co-infection of Ad/CMV-EGFP and a control virus Ad/SP-C-shCon expressing unrelated shRNA failed to reduce the EGFP expression (FIG. 2B b). The quantitation by RT-PCR and Western blotting indicated that SP-C-driven shEGFP decreased the mRNA and protein levels of EGFP by 74% and 81%, respectively, but did not alter □-actin expression (FIG. 2D and FIG. 2E). Another vector, Ad/SP-C-shEGFP-pA, containing 0.45-kb SV40 poly A instead of the 66 bp minimal poly A, did not show a significant inhibition of EGFP expression under the same conditions (data not shown), consistent with a previous report on the CMV promoter (3). To examine whether SP-C-driven shRNA is specific to type II cells, we repeated the experiment above with a number of cell lines that do not express SP-C. No reductions of EGFP mRNA and protein levels were observed in a rat lung epithelial cell line, L2 cells infected with Ad/SP-C-shEGFP as determined by RT-PCR, Western blotting, and a fluorescence microscopy (FIG. 2C, D and E). Similar results were obtained with two mouse fibroblast cell lines, NIH 3T3 and L929 (data not shown).

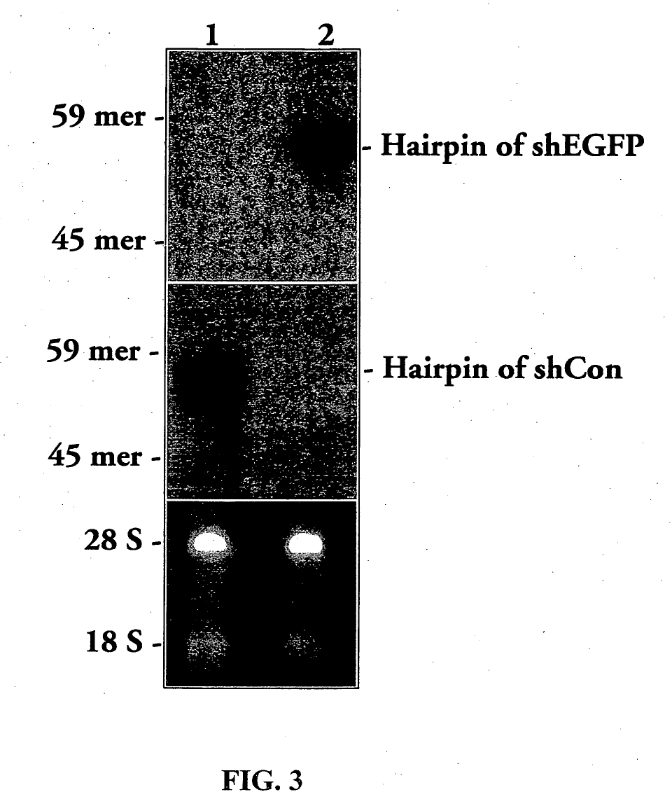

Northern blotting analysis showed that Ad/SPC-shEGFP or Ad/SPC-shCon infected type II cells expressed a ˜57 base pair RNA specific to shEGFP or shCon sequences (FIG. 3), consistent with the predicted size of the transcripted shRNA, further indicating that SP-C promoter can be used to express shRNAs and silence gene expression specifically in type II cells.

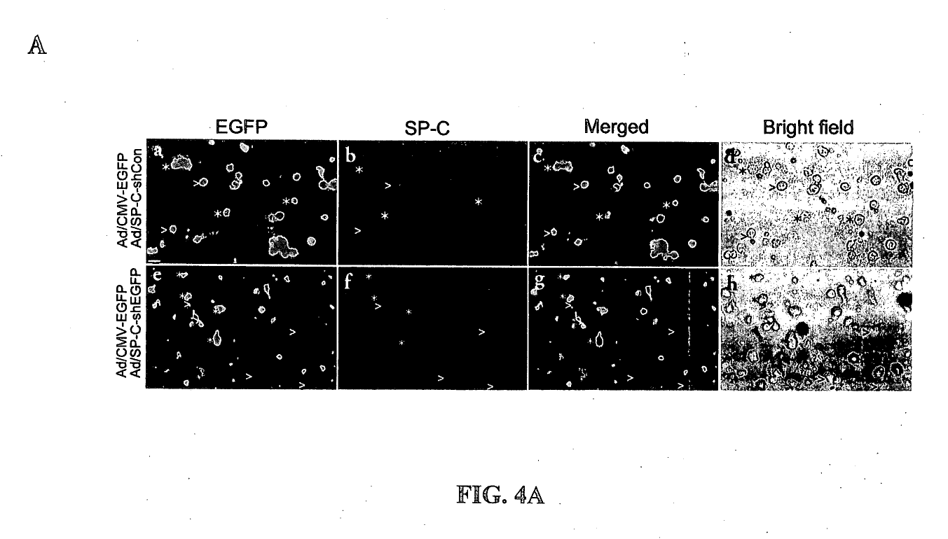

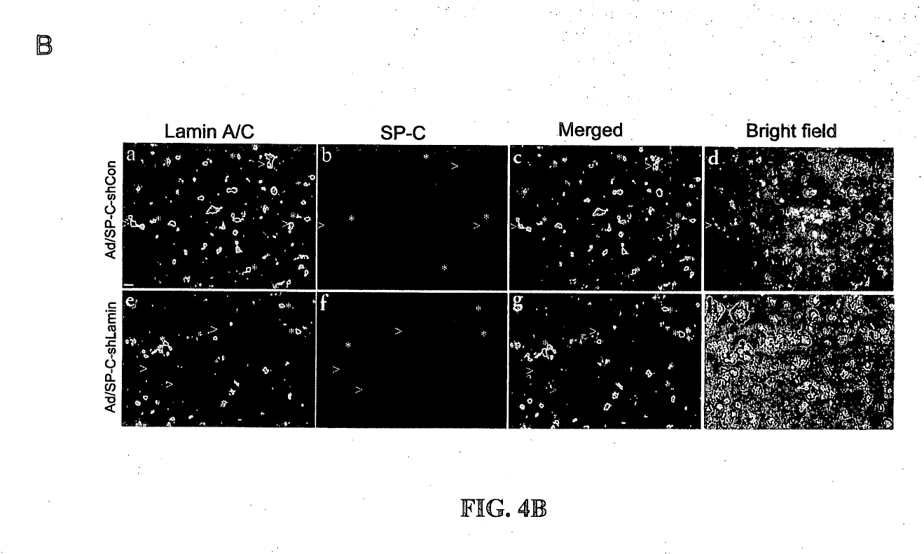

About 40 different cell types exist in the lungs. To further demonstrate the specificity of SP-C-driven shRNA, we prepared a lung cell mixture from the elastase-digested rat lungs (13) and used it for gene silencing. The cell preparation contains alveolar type I and type II cells, Clara cells, ciliated airway epithelial cells, fibroblasts, macrophages, and lymphocytes. The mixed cells were cultured on an air-liquid cell culture system as described above and infected with Ad/CMV-EGFP in the presence of Ad/SP-C-shEGFP or Ad/SP-C-shCon. EGFP fluorescence was monitored with a fluorescence microscope, and type II cells were identified by immunostaining using anti-SP-C antibodies. SP-C expression was taken into consideration to ascertain the gene silencing occurred only in type II cells and not in other cells. In the Ad/SP-C-shEGFP-treated group, EGFP signals was markedly decreased in a majority of type II cells (arrows, FIG. 4A e-h), but not in non-type II cells (stars). We quantified the results by counting the number of type II cells (red) with or without EGFP fluorescence (green). The data revealed that the silencing of EGFP occurred in ˜73% of the type II cells (FIG. 4C). The lack of a complete inhibition may be, in part, due to a high level of EGFP expression directed by a strong CMV promoter. In the control group with the Ad/SP-C-shCon, all the cells, including type II cells (arrows, FIG. 4A a-d) and non-type II cells (stars), were positive for the EGFP signals, suggesting that the unrelated siRNA control adenovirus failed to reduce the GFP level in the type II cells and non-type II cells.

The ability of shRNAs controlled by SP-C promoter to work on endogenous genes was tested. Two endogenous genes with diverse functions and different cellular locations were selected, lamin A/C and annexin A2. Lamin A/C is a nuclear membrane protein involved in the organization of nuclear architecture (20). Annexin A2 is a cytosolic Ca²⁺-dependent phospholipid-binding protein and plays an important role in the membrane fusion during the exocytosis of lamellar bodies from alveolar epithelial type II cells (21). The siRNA sequence, targeted to the coding region of 607 to 625 of rat lamin A/C gene, was chosen based on the previous reports on human lamin A/C gene, (2;4). In this region, there is one base difference between rat and human sequences, which we switched to the rat sequence (C⁶¹¹→T⁶¹¹ ). A mixed lung cell culture was infected with Ad/SP-C-shLamin or Ad/SP-C-shCon. After a 4-day culture, the cells were double-labeled with anti-lamin A/C and anti-SP-C antibodies to determine the protein expression level of lamin A/C and to identify alveolar type II cells, respectively. As shown in FIG. 4B a-d, Lamin A/C was expressed at a similar level in all the different cells when they were infected by Ad/SP-C-shCon adenovirus. However, the expression of lamin A/C was specifically reduced in type II cells (arrows, FIG. 4B e-h), but not in non-type II cells (stars) by Ad/SP-C-shLamin adenovirus. Quantified data by counting type II cells (red) with or without reduced lamin A/C expression (green) revealed that the silence of Lamin A/C occurred in ˜44% of type II cells.

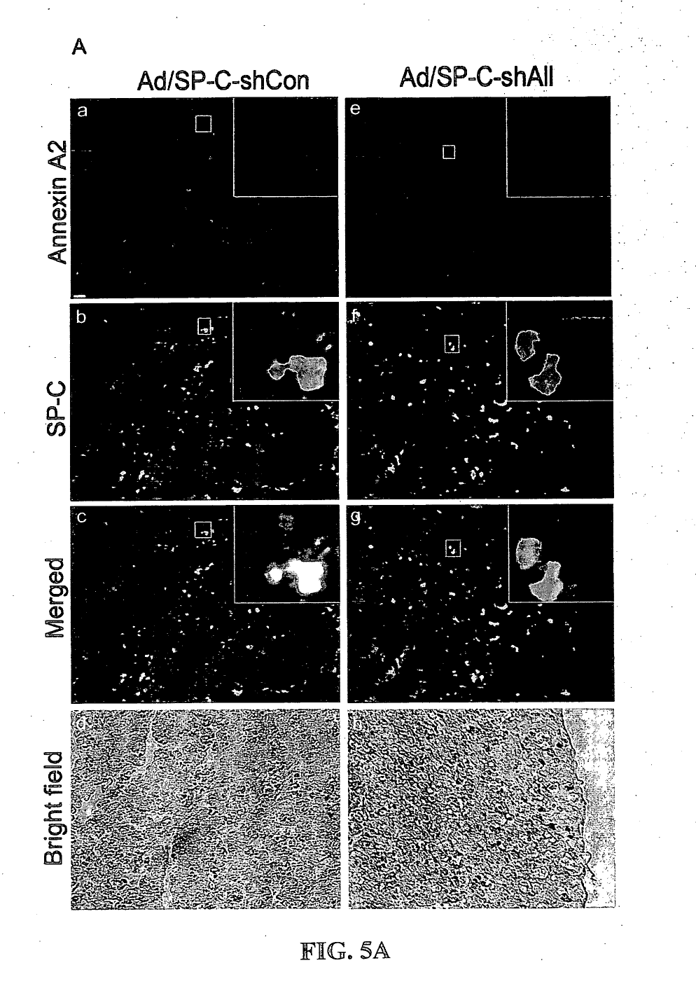

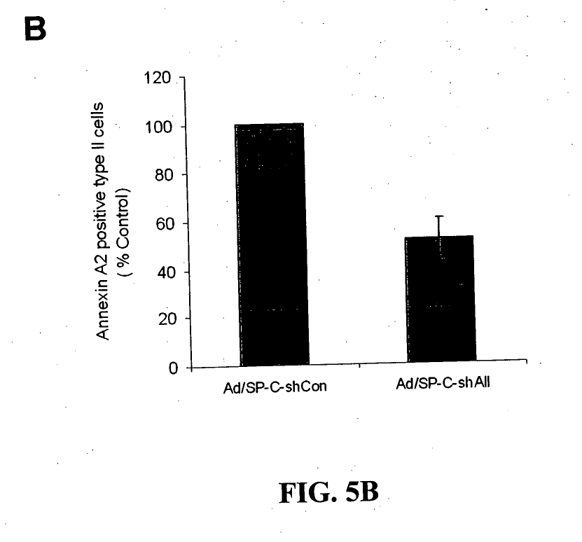

Dissection of lung cells from rat lung tissues and the cell isolation procedure may alter cell function and activate gene expression. Therefore, an in vitro model of neonatal rat lung organ culture was used to further test the specific silencing of an endogenous gene, annexin A2. This organ culture system maintains the cellular architecture and thus intracellular contacts and communications (15). We have previously screened six in vitro transcribed siRNA sequences targeted to different regions of the rat annexin A2 gene and found that the siRNA sequence targeted to 129-147 nucleotide was the most effective in silencing annexin A2 protein expression in type II cells (22). This annexin A2 siRNA sequence was used in the present study to construct an adenoviral vector, Ad/SP-C-shAII. Double-labeling with anti-annexin A2 and SP-C antibodies was used to determine annexin A2 protein expression level (red) and to identify type II cells (green), respectively. The infection of the neonatal lung organ culture with Ad/SP-C-shAII generated green-positive (type II cells) and red-negative (annexin A2 expression level) cells (FIG. 5A e-h), suggesting the silencing of annexin A2 in type II cells. In the siRNA control-treated group, we observed green-positive and red-positive cells, indicating a high expression of annexin A2 in type II cells (FIG. 5A a-d). A clear difference can be seen in the merged images, in which the yellow spots in the siRNA control group and the green spots in the annexin A2 siRNA group represent the expression and silencing of annexin A2 in type II cells, respectively. The cell counting revealed that the cells showing both SP-C and annexin A2 staining were reduced by 48% when compared to the controls (FIG. 5B). In both groups, some of the cells showed green-negative and red-positive cells, indicating no silencing of annexin A2 in the non-type II cells. The results suggest that the observed RNAi effect on endogenous annexin A2 gene in lung organ culture is cell- and sequence-specific.

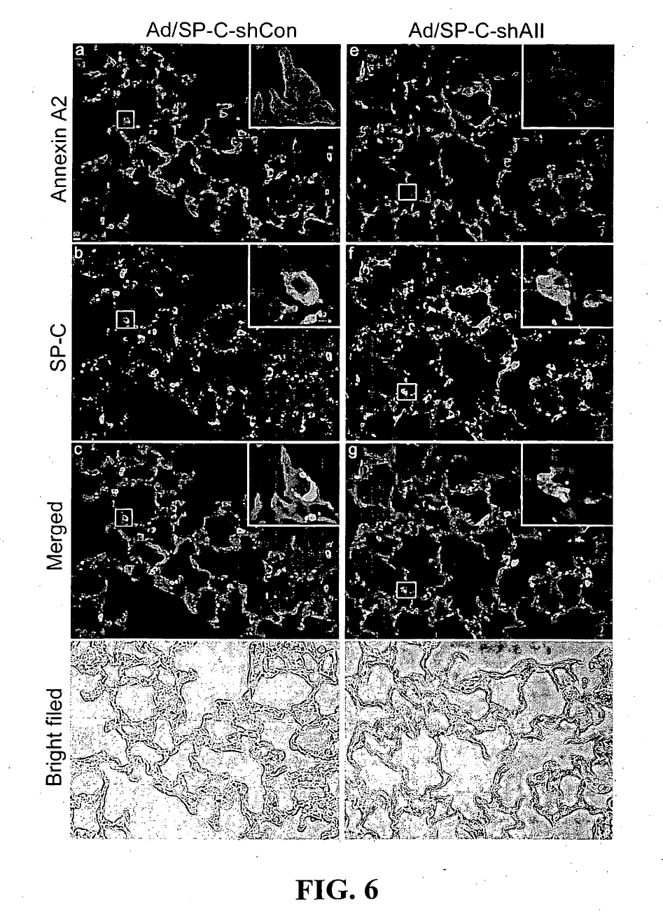

Finally, to examine whether SP-C-driven shRNA works in vivo, we directly delivered the adenoviral vector, Ad/SP-C-shAII, into rat lungs and determined annexin A2 expression in type II cells. The virus was delivered to rat lungs intrabronchially. Surfactant and protamine were included to enhance the efficiency of adenovirus-mediated siRNA expression, and corticosteroids were used to inhibit inflammation and minimize virus-related toxicity (17;18). Rats were sacrificed and the lungs infused with 4% (w/v) formaldehyde 5 days after delivery. The tissue section was double-stained with anti-annexin A2 and anti-SP-C antibodies. In the Ad/SP-C-shAII-treated group, we found that annexin A2 was silenced in type II cells (green-positive and red-negative cells) (FIG. 6 e-h). However, the expression of annexin A2 in non-type II cells was evident (green-negative and red-positive cells). In contrast, both green- and red-positive cells (annexin A2 expressed in type II) and green-negative and red-positive cells (annexin A2 expressed in non-type II) were seen in the control group (FIG. 6 a-d). The number of type II cells showing silencing of annexin A2 was ˜15%. Although the current results showed a low silencing efficiency, our studies are promising considering the low infection efficiency in the in vivo studies. We estimated 20-30% infection efficiency in our studies by delivering Ad/CMV-EGFP virus, digesting lungs and counting EGFP-positive lungs. This experiment demonstrates that SP-C promoter drives siRNA expression and silences gene expression in a cell-specific manner in animals in vivo.

The most commonly used plasmids for expressing siRNAs in cells contain RNA polymerase III-based promoters such as U6 and H1 or RNA polymerase II promoters such as CMV (3-6). Although those promoters have a wide applicability in cell systems in vitro, their lack of cell specificity limits their usage in vivo. We have demonstrated that SP-C-driven shRNA expression effectively and specifically silences exogenous and endogenous gene expression in type II lung cells using in vitro cell and organ culture as well as in vivo. The current studies, therefore, establish proof-of-principle for using a cell-specific promoter to depress gene expression in a particular type of cell. This provides an efficient strategy in targeting and silencing a specific gene in vivo in one type of cell without interfering with other cell systems. For example, in order to target an oncogene in cancer cells, but not normal cells, a telomerase reverse transcriptase (TERT) promoter (23) would be used to drive shRNA expression in cancer cells. Considerable interest has developed in the potential for RNAi therapy. As a therapeutic agent, there is a great need for delivering siRNA to and thus silencing a gene in a particular type of target cell. This strategy may have a significant impact on RNAi therapy.

### REFERENCES

1. Hannon, G. J. (2002) RNA interference. Nature, 418, 244-251.
2. Elbashir, S. M., Harborth, J., Lendeckel, W., Yalcin, A., Weber, K. and Tuschl, T. (2001) Duplexes of 21-nucleotide RNAs mediate RNA interference in cultured mammalian cells. Nature, 411, 494-498.

3. Xia, H., Mao, Q., Paulson, H. L. and Davidson, B. L. (2002) siRNA-mediated gene silencing in vitro and in vivo. Nat Biotechnol, 20, 1006-1010.
4. Sui, G., Soohoo, C., Affar, e. B., Gay, F., Shi, Y. and Forrester, W. C. (2002) A DNA vector-based RNAi technology to suppress gene expression in mammalian cells. Proc Natl Acad Sci USA, 99, 5515-5520.
5. Brummelkamp, T. R., Bernards, R. and Agami, R. (2002) A system for stable expression of short interfering RNAs in mammalian cells. Science, 296, 550-553.
6. Gou, D., Jin, N. and Liu, L. (2003) Gene silencing in mammalian cells by PCR-based short hairpin RNA. FEBS Lett., 548, 113-118.
7. Robinson, R. (2004) RNAi Therapeutics: How Likely, How Soon? PLoS.Biol., 2, E28.
8. Kasim, V., Miyagishi, M. and Taira, K. (2004) Control of siRNA expression using the Cre-loxP recombination system. Nucleic Acids Res, 32, e66.
9. Tichelaar, J. W., Lu, W. and Whitsett, J. A. (2000) Conditional expression of fibroblast growth factor-7 in the developing and mature lung. J Biol Chem, 275, 11858-11864.
10. Engelhardt, J. F., Yang, Y., Stratford-Perricaudet, L. D., Allen, E. D., Kozarsky, K., Perricaudet, M., Yankaskas, J. R. and Wilson, J. M. (1993) Direct gene transfer of human CFTR into human bronchial epithelia of xenografts with E1-deleted adenoviruses. Nat. Genet., 4, 27-34.
11. He, T. C., Zhou, S., da Costa, L. T., Yu, J., Kinzler, K. W. and Vogelstein, B. (1998) A simplified system for generating recombinant adenoviruses. Proc Natl Acad Sci USA, 95, 2509-2514.
12. Liu, L., Wang, M., Fisher, A. B. and Zimmerman, U. J. P. (1996) Involvement of annexin II in exocytosis of lamellar bodies from alveolar epithelial type II cells. Am. J. Physiol., 270, L668-L676.
13. Chen, J. W., Chen, Z., Narasaraju, T., Jin, N. and Liu, L. (2004) Isolation of hightly pure alveolar epithelial type I and type II cells from rat lungs. Lab Invest, 84, 727-735.
14. Mason, R. J., Lewis, M. C., Edeen, K. E., McCormick-Shannon, K., Nielsen, L. D. and Shannon, J. M. (2002) Maintenance of surfactant protein A and D secretion by rat alveolar type II cells in vitro. Am J Physiol Lung Cell Mol Physiol, 282, L249-L258.
15. Chetty, A. and Nielsen, H. C. (2002) Regulation of cell proliferation by insulin-like growth factor 1 in hyperoxia-exposed neonatal rat lung. Mol. Genet. Metab, 75, 265-275.
16. Jou, I. M., Tsai, Y. T., Tsai, C. L., Wu, M. H., Chang, H. Y. and Wang, N. S. (2000) Simplified rat intubation using a new oropharyngeal intubation wedge. J Appl. Physiol, 89, 1766-1770.
17. Lin, T., Gu, J., Zhang, L., Davis, J. J., Huang, X., Cabbini, G., Ji, L. and Fang, B. (2003) Enhancing adenovirus-mediated gene transfer in vitro and in vivo by addition of protamine and hydrocortisone. J. Gene Med., 5, 868-875.
18. Richard, J. C., Factor, P., Welch, L. C. and Schuster, D. P. (2003) Imaging the spatial distribution of transgene expression in the lungs with positron emission tomography. Gene Ther., 10, 2074-2080.
19. Narasaraju, T. A., Jin, N., Narendranath, C. R., Chen, Z., Gou, D. and Liu, L. (2003) Protein nitration in rat lungs during hyperoxia exposure: a possible role of myeloperoxidase. Am. J. Physiol Lung Cell Mol. Physiol, 285, L1037-L1045.
20. Wilson, K. L. (2000) The nuclear envelope, muscular dystrophy and gene expression. Trends Cell Biol., 10, 125-129.
21. Chattopadhyay, S., Sun, P., Wang, P., Abonyo, B., Cross, N. L. and Liu, L. (2003) Fusion of lamellar body with plasma membrane is driven by the dual action of annexin II tetramer and arachidonic acid. J. Biol. Chem., 278, 39675-39683.
22. Gou, D., Wang, P., Jin, N. and Liu, L. (2004) Silencing of annexin II in primary culture of alveolar epithelial type II cells. Annexins, in press.
23. Gu, J., Zhang, L., Huang, X., Lin, T., Yin, M., Xu, K., Ji, L., Roth, J. A. and Fang, B. (2002) A novel single tetracycline-regulative adenoviral vector for tumor-specific Bax gene expression and cell killing in vitro and in vivo. Oncogene, 21, 4757-4764.

We claim:

1. A method for the treatment of a condition caused or exacerbated by expression of at least one specific gene in a specific type of cell or tissue, comprising the step of
providing to a patient in need thereof a composition which includes a nucleic acid construct, said nucleic acid construct comprising:
i) at least one expressible DNA sequence encoding RNA capable of forming a dsRNA structure, wherein a nucleotide sequence of a portion of said dsRNA structure is identical to a nucleotide sequence of a portion of mRNA encoded by said at least one specific gene; and
ii) at least one promoter sequence operationally linked to said at least one expressible DNA sequence, wherein said at least one promoter sequence is active only in said specific type of cell;
wherein said nucleic acid construct is provided in an amount sufficient to decrease translation of said mRNA encoded by said specific gene.

2. The method of claim 1, wherein said specific type of cell or tissue is selected from the group consisting of lung epithelial type II cells, lung epithelial type I cells, lung Clara cells, lung epithelial cells, cells, lung airway surface epitheilium, lung endothelial cells, liver, muscle, oligodendrocytes, glial cells, neurons, tumour endothelium, cancer cells, lung cancer cells, breast cancer cells, colon cancer cells, hepatocellular carcinoma (HCC) cells, melanoma cells, prostate cells, osteosarcoma cells, ovarian cancer cells, cervical cancer cells, and solid tumour cells.

3. The method of claim 1, wherein said at least one promoter sequence is selected from the group consisting of SP-C and SP-B promoter, aquaporin 5 promoter, CCSP promoter, cytokeratin 18 (K18) promoter, vascular endothelial growth factor receptor type-1 (flt-1) promoter, FOXJ1 promoter, Tie2 promoter, Pre-proendothelin-1 (PPE-1) promoter, Albumin promoter MCK promoter, Myelin basic protein promoter, Glial fibrillary acidic protein promoter, NSE promoter, KDR promoter, E-selectin promoter, endoglin promoter, telomerase reverse transcriptase promoter, carcinoembryonic antigen (CEA) promoter, alpha-ftoprotein (AFP) promoter, ErbB2 promoter,tyrosinase gene promoter, prostate-specific antigen (PSA) promoter, muc-1 promoter, osteocalcin promoter, secretory leukoprotease inhibitor, and HRE promoter.

4. The method of claim 1, wherein said at least one specific gene is selected from the group consisting of fBCR/ABL fusion protein, K-RAS, H-RAS, bcl-2, Bax, FGF-4, Skp-2, CEACAM6, MMP-9, Rho, spingosine-1 phosphate-R, EGF receptor, EphA2, focal adhesion kinase, surviving, colony-stimulating factor, Wnt, PI3 kinase, Cox-2, H-Ras, CXCR4, BRAF, Brk, PKC-alpha, telomerase, myc, ErbB-2, cyclin D1, TGF-alpha, Akt2,3, 6 4 integrin, EPCAM receptor, androgen receptor, MDR, viral genes, host receptor genes, HB surface antigen, viral gene CD46, viral gene PP1, VEGF, VEGF receptor,TGF-beta receptor, SOD1, Beta-secretase (BACE1), SCCMS, Huntingin, Ataxin 1, TGF-alpha, TGF-beta, Smad, CFTR, MIP-2, keratinocyte-derived chemokine (KC), AGRP, Apo B, TNF-alpha, Gap junction beta2.

5. The method of claim 1, wherein said specific type of cell is a lung cell.

6. The method of claim 1 wherein said specific type of cell is a cancer cell.

7. The method of claim 1, wherein said dsRNA structure is shRNA.

8. The method of claim 1, wherein said construct includes an adenoviral vector.

9. A nucleic acid construct comprising,
i) at least one expressible DNA sequence encoding RNA capable of forming a dsRNA structure, wherein a nucleotide sequence of a portion of said dsRNA structure is identical to a nucleotide sequence of a portion of an mRNA of interest; and
ii) at least one promoter sequence operationally linked to said at least one expressible DNA, wherein said at least one promoter sequence is active only in a specific type of cell.

10. The nucleic acid construct of claim 9, wherein said specific type of cell or tissue is selected from the group consisting of lung epithelial type II cells, lung epithelial type I cells, lung Clara cells, lung epithelial cells, cells, lung airway surface epitheilium, lung endothelial cells, liver, muscle, oligodendrocytes, glial cells, neurons, tumour endothelium, cancer cells, lung cancer cells, breast cancer cells, colon cancer cells, hepatocellular carcinoma (HCC) cells, melanoma cells, prostate cells, osteosarcoma cells, ovarian cancer cells, cervical cancer cells, and solid tumour cells.

11. The nucleic acid construct of claim 9, wherein said at least one promoter sequence is selected from the group consisting of SP-C and SP-B promoter, aquaporin 5 promoter, CCSP promoter, cytokeratin 18 (K18) promoter, vascular endothelial growth factor receptor type-1 (flt-1) promoter, FOXJ1 promoter, Tie2 promoter, Pre-proendothelin-1 (PPE-1) promoter, Albumin promoter MCK promoter, Myelin basic protein promoter, Glial fibrillary acidic protein promoter, NSE promoter, KDR promoter, E-selectin promoter, endoglin promoter, telomerase reverse transcriptase promoter, carcinoembryonic antigen (CEA) promoter, alpha-ftoprotein (AFP) promoter, ErbB2 promoter,tyrosinase gene promoter, prostate-specific antigen (PSA) promoter, muc-1 promoter, osteocalcin promoter, secretory leukoprotease inhibitor, and HRE promoter.

12. The nucleic acid construct of claim 9, wherein said at least one specific gene is selected from the group consisting of fBCR/ABL fusion protein, K-RAS, H-RAS, bcl-2, Bax, FGF-4, Skp-2, CEACAM6, MMP-9, Rho, spingosine-1 phosphate-R, EGF receptor, EphA2, focal adhesion kinase, surviving, colony-stimulating factor, Wnt, PI3 kinase, Cox-2, H-Ras, CXCR4, BRAF, Brk, PKC-alpha, telomerase, myc, ErbB-2, cyclin D1, TGF-alpha, Akt2,3, 6 4 integrin, EPCAM receptor, androgen receptor, MDR, viral genes, host receptor genes, HB surface antigen, viral gene CD46, viral gene PP1, VEGF, VEGF receptor,TGF-beta receptor, SOD1, Beta-secretase (BACE1), SCCMS, Huntingin, Ataxin 1, TGF-alpha, TGF-beta, Smad, CFTR, MIP-2, keratinocyte-derived chemokine (KC), AGRP, Apo B, TNF-alpha, Gap junction beta2.

13. The nucleic acid construct of claim 9, wherein said specific type of cell is a lung cell.

14. The nucleic acid construct of claim 13, wherein said lung cell is an alveolar Type II cell.

15. The nucleic acid construct of claim 9, wherein said promoter is SP-C.

16. The nucleic acid construct of claim 9, wherein said dsRNA structure is shRNA.

17. The nucleic acid construct of claim 9, wherein said nucleic acid construct includes an adenoviral vector.

18. A method of reducing expression of at least one specific gene by decreasing translation of mRNA encoded by said at least one specific gene in a specific type of cell or tissue, comprising the step of
providing to said specific type of cell or tissue a nucleic acid construct comprising
i) at least one expressible DNA sequence encoding RNA capable of forming a dsRNA structure, wherein a nucleotide sequence of a portion of said dsRNA structure is identical to a nucleotide sequence of a portion of mRNA encoded by said specific gene; and
ii) at least one promoter sequence operationally linked to said at least one expressible DNA, wherein said at least one promoter sequence is active only in said specific type of cell or tissue.

19. The method of claim 18, wherein said method is carried out in vivo in a patient in need thereof.

20. The method of claim 18, wherein said specific type of cell or tissue is selected from the group consisting of lung epithelial type II cells, lung epithelial type I cells, lung Clara cells, lung epithelial cells, cells, lung airway surface epitheilium, lung endothelial cells, liver, muscle, oligodendrocytes, glial cells, neurons, tumour endothelium, cancer cells, lung cancer cells, breast cancer cells, colon cancer cells, hepatocellular carcinoma (HCC) cells, melanoma cells, prostate cells, osteosarcoma cells, ovarian cancer cells, cervical cancer cells, and solid tumour cells.

21. The method of claim 18, wherein said at least one promoter sequence is selected from the group consisting of SP-C and SP-B promoter, aquaporin 5 promoter, CCSP promoter, cytokeratin 18 (K18) promoter, vascular endothelial growth factor receptor type-1 (flt-1) promoter, FOXJ1 promoter, Tie2 promoter, Pre-proendothelin-1 (PPE-1) promoter, Albumin promoter MCK promoter, Myelin basic protein promoter, Glial fibrillary acidic protein promoter, NSE promoter, KDR promoter, E-selectin promoter, endoglin promoter, telomerase reverse transcriptase promoter, carcinoembryonic antigen (CEA) promoter, alpha-ftoprotein (AFP) promoter, ErbB2 promoter,tyrosinase gene promoter, prostate-specific antigen (PSA) promoter, muc-1 promoter, osteocalcin promoter, secretory leukoprotease inhibitor, and HRE promoter.

22. The method of claim 18, wherein said at least one specific gene is selected from the group consisting of fBCR/ABL fusion protein, K-RAS, H-RAS, bcl-2, Bax, FGF-4, Skp-2, CEACAM6, MMP-9, Rho, spingosine-1 phosphate-R, EGF receptor, EphA2, focal adhesion kinase, surviving, colony-stimulating factor, Wnt, PI3 kinase, Cox-2, H-Ras, CXCR4, BRAF, Brk, PKC-alpha, telomerase, myc, ErbB-2, cyclin D1, TGF-alpha, Akt2,3, 6 4 integrin, EPCAM receptor, androgen receptor, MDR, viral genes, host receptor genes, HB surface antigen, viral gene CD46, viral gene PP1, VEGF, VEGF receptor,TGF-beta receptor, SOD 1, Beta-secretase (BACE1), SCCMS, Huntingin, Ataxin 1, TGF-alpha, TGF-beta, Smad, CFTR, MIP-2, keratinocyte-derived chemokine (KC), AGRP, Apo B, TNF-alpha, Gap junction beta2.

23. The method of claim 18, wherein said specific type of cell is a lung cell.

24. The method of claim 23, wherein said lung cell is an alveolar Type II cell.

25. The method of claim 18, wherein said at least one promoter is SP-C.

26. The method of claim 18, wherein said dsRNA structure is shRNA.

27. The method of claim 18, wherein said nucleic acid construct includes an adenoviral vector.


SEQUENCE LISTING (16 sequences)

SEQ ID NO: 1 - DNA, 40 bp, Artificial
  Synthetic oligonucleotide primer
cacctcgagatcgatgaagactgctgctctctaccacgtt

SEQ ID NO: 2 - DNA, 44 bp, Artificial
  Synthetic oligonucleotide primer
ttcgaacgcgtaattcgagctcggtacccggggatcagacatga

SEQ ID NO: 3 - DNA, 66 bp, Artificial
  Synthetic oligonucleotide primer
aattcgaattcaataaaggatcctttattttcattggatccgtgtgttggttttttgtgtctcgag

SEQ ID NO: 4 - DNA, 66 bp, Artificial
  Synthetic oligonucleotide primer
gatcctcgagacacaaaaaaccaacacacggatccaatgaaaataaaggatcctttattgaattcg

SEQ ID NO: 5 - DNA, 58 bp, Artificial
  Synthetic oligonucleotide primer
tcgaccacaagctggagtacaactacttcaagagagtagttgtactccagctgtgttg

SEQ ID NO: 6 - DNA, 59 bp, Artificial
  Synthetic oligonucleotide primer
aattcaacacaagctggagtacaactactctcttgaagtagttgtactccagcttgtgg

SEQ ID NO: 7 - DNA, 55 bp, Artificial
  Synthetic oligonucleotide primer
tcgacctggatttccagaagaacattcaagagatgttcttctggaaatccagttg

SEQ ID NO: 8 - DNA, 55 bp, Artificial
  Synthetic oligonucleotide primer
aattcaactggatttccagaagaacatctcttgaatgttcttctggaaatccagg

SEQ ID NO: 9 - DNA, 57 bp, Artificial
  Synthetic oligonucleotide primer
tcgaccgcattgaaacagcaatcaagttcaagagacttgattgctgtttcaatgttg

SEQ ID NO: 10 - DNA, 57 bp, Artificial
  Synthetic oligonucleotide primer
aattcaacattgaaacagcaatcaagtctcttgaacttgattgctgtttcaatgcgg

SEQ ID NO: 11 - DNA, 55 bp, Artificial
  Synthetic oligonucleotide primer
tcgacttctccgaacgtgtcacgtttcaagagaacgtgacacgttcggagaattg

SEQ ID NO: 12 - DNA, 50 bp, Artificial
  Synthetic oligonucleotide primer
aattctccgaacgtgtcacgttctcttgaaacgtgacacgttcggagaag

SEQ ID NO: 13 - DNA, 20 bp, Artificial
  Synthetic oligonucleotide primer
tgccacctacggcaagctga

SEQ ID NO: 14 - DNA, 20 bp, Artificial
  Synthetic oligonucleotide primer
tcgatgttgtggcggatctt

SEQ ID NO: 15 - DNA, 27 bp, Artificial
  Synthetic oligonucleotide primer
ggcattgtaaccaactgggacgatatg

SEQ ID NO: 16 - DNA, 22 bp, Artificial
  Synthetic oligonucleotide primer
ttcatggatgccacaggattcc